(19) United States
(12) Patent Application Publication
(10) Pub. No.: US 2024/0285692 A1
(43) Pub. Date: Aug. 29, 2024

(54) USE OF DEATH LIGANDS ON HEMATOPOIETIC STEM AND PROGENITOR CELLS AND MESENCHYMAL STROMAL CELLS FOR CANCER THERAPY

(71) Applicant: COCYTUS LTD., Tel Aviv-Yafo (IL)

(72) Inventor: Nadir ASKENASY, Tel Aviv-Yafo (IL)

(73) Assignee: COCYTUS LTD., Tel Aviv-Yafo (IL)

(21) Appl. No.: 18/567,926

(22) PCT Filed: Jun. 7, 2022
(86) PCT No.: PCT/IL2022/050605

§ 371 Date: Dec. 7, 2023

Related U.S. Application Data
(60) Provisional application No. 63/197,530, filed on Jun. 7, 2021.

(51) Int. Cl. A61K 35/28; A61K 38/00; A61P 35/00; C07K 14/705; C12N 5/0775; C12N 5/0789

(52) U.S. Cl. CPC A61K 35/28; A61P 35/00; C07K 14/70575; C12N 5/0647; C12N 5/0663; A61K 38/00; C12N 2510/00

(57) ABSTRACT

Methods of producing modified cells comprising providing a sample comprising hematopoietic stem or progenitor cells (HSPC) or mesenchymal stromal cells (MSC) and adsorbing a death ligand protein to a plasma membrane of the HSPCs or MSCs are provided. Modified cells, and HSPCs and MSCs having adhered thereto an exogenous death ligand protein as well as compositions comprising those cells and their use in treating cancer are also provided.

### CROSS REFERENCE TO RELATED APPLICATIONS

This application claims the benefit of priority under 35 U.S.C. § 119(e) of U.S. Provisional Patent Application No. 63/197,530, filed Jun. 7, 2021, entitled “USE OF HEMATOPOIETIC STEM AND PROGENITOR CELLS AND MESENCHYMAL STROMAL CELLS FOR CANCER THERAPY”, which is incorporated by reference in its entirety.

### FIELD OF INVENTION

The present invention is in the field of cancer therapeutics.

### BACKGROUND OF THE INVENTION

### Cancer Therapy

Physiological immune surveillance is responsible for elimination of cancer cells early after malignant transformation. Immunotherapy aims to harness this immune mechanism against cancer by supplementation of cytokines, chemokines, proteins and antibodies that invigorate the activity of lymphocytes against tumors. For example, soluble factors include stimulatory cytokines such as interleukin-2 and interferon-α, and modulation of costimulatory signals by antibodies that neutralize inhibitory molecules such as cytotoxic T lymphocyte antigen-4 and programed death-1 ligand, along neutralization of suppressor (regulatory) T cells. Immunotherapy through enhanced activity of patients' own immune system against cancer has shown promising results, however is associated with quite severe side effects of non-selective immune stimulation. Evidently, this therapeutic approach can be not incorporated within standard radiochemotherapy protocols because competence of patient's immune system is an obligatory prerequisite.

Another approach uses autologous and allogeneic cells, which are extracted, amplified and adoptively transferred, including ex vivo stimulation of dendritic cells and chemokine-activated killer cells, expansion of tumor-infiltrating lymphocytes, and T cell engineering to overexpress chimeric receptors selective for tumor antigens (CAR-T). The primary implementation of transient cellular immunotherapy has been achieved by infusion of lymphocytes into immunocompetent patients without preparatory conditioning, in order to prevent evolution of GvHD. While autologous lymphocytes are generally less potent, allogeneic and haploidentical lymphocytes are acutely rejected within few days, limiting the therapeutic window to a quite narrow period of time.

The most potent procedure to enforce immune surveillance is induction of mixed hematopoietic chimerism by means of allogeneic bone marrow transplantation. Transplants consist either of autologous immunohematopoietic reconstitution following aggressive radiochemotherapy to ensure recovery of functional hematopoietic progenitors, or replacement by allogeneic hematopoietic stem and progenitor cells (HSPC) with the distinct advantage of generating potent graft versus tumor reactions (GvT). Unfortunately, these complex procedures are accompanied by significant morbidity due to extended periods of susceptibility to infections, end organ toxicity, failure to engraft and potentially lethal graft versus host disease (GvHD).

The end point of immunotherapy involves the interaction between sensitized immune cells that use one of the killing mechanisms to induce apoptosis in the tumor cell. Major mechanisms consist of introduction of granzyme into the target cancer cell by means of membrane permeabilization with perforin (perforin/granzyme), radical oxygen species (ROS) or presentation of ligands of the tumor necrosis factor (TNF) superfamily.

### Tumor Necrosis Factor (TNF) Superfamily

Receptor-ligand interactions of the TNF superfamily are executioner mechanisms of death through induction of apoptosis in parenchymal, stromal, hematopoietic, immune and tumor cells. For example, all immune cells are submitted to negative regulation by activation-induced cell death (AICD) due to acquired susceptibility to apoptosis along differentiation and maturation, and acute upregulation of the TNF family receptors following activation. This pivotal mechanism of elimination by death is essential in negative regulation of expanding immune cells and of terminally differentiated hematopoietic clones, thus ensuring hematopoietic heterogeneity.

### Hematopoietic Progenitors are Resistant to Apoptotic Signaling

Although upregulation of the TNF family receptors is a conserved response of the immune and hematopoietic systems to stress and activation, the consequences are different: stimulation of progenitor development and lethal downregulation of the differentiated progeny. This divergent mechanism is made possible by the inherent resistance of murine and human hematopoietic stem and progenitor cells to apoptotic signaling mediated by the Fas, TNF and TNF-related apoptosis-inducing ligand (TRAIL) receptors, which is regulated at the transcriptional level. Thus, in variance from the mature progeny, apoptosis-resistant hematopoietic stem and progenitor cells are transduced with growth signals by the TNF family. These receptor/ligand interactions of the TNF superfamily couple injury and inflammation to activity of hematopoietic progenitors in order to fuel immune reactions.

### Sensitivity of Malignant Cells to Apoptotic Signaling

Most hematological malignancies, including leukemia and lymphoma, are sensitive to apoptotic signaling by the TNF family receptors and can be therefore eliminated by targeted delivery of death ligands. Likewise, a number of solid tumors display sensitivity to apoptosis including prevalent diseases such as colon, lung, prostate, ovary and breast carcinoma, and rare tumors for which there is no effective therapy such as osteosarcoma, neuroblastoma, glioblastoma, melanoma, uroepithelial, hepatic and cholangic carcinomas. More successful implementation of hematopoietic cell transplants in hematological malignancies is likely caused by ubiquitous expression of minor histocompatibility antigens (miHA) in leukemia and lymphoma cells that serve as targets of immune attack, enhanced by major histocompatibility antigen (MHC) mismatch. The TNF family receptor ligand interactions are also involved in mechanisms of tumor defense and growth. For example, cancer cells and tumor stroma express ligands of the TNF superfamily for self-defense through induction of apoptosis in infiltrating immune cells. In addition, the Fas/FasL interaction and particularly the soluble ligand shed from the surface of cells by matrix metalloproteinases participates in diverse mechanisms enhancing tumor growth and metastatic spread.

Apoptotic activity of FasL requires trimerization of the Fas receptor, optimally attained by the membrane-bound ligand. A number of fusion proteins have been composed for selective delivery of FasL by targeting antigens of malignant cells, such as leukemia inhibitory factor receptor and a glial membranal protein for targeting intracranial neoplasms. Other bispecific antibodies have been designed for selective delivery of FasL with affinity to lymphocyte markers such as CD7 in T cells and CD20 in B lymphocytes. An interesting design of fusion molecules aims to simultaneously inhibit the suppressor molecules CD40 and CTLA-4 and deliver apoptotic signals by virtue of a conjugated FasL moiety. Other approaches have targeted tumor stroma to deliver apoptotic signals to malignant cells, such as coupling FasL to fibroblast activating protein and a glycoprotein antibody. Furthermore, FasL conjugation to a collagen binding domain of adipocytes has been quite effectively applied against leukemia and by stereotactic local introduction into glioblastoma.

### SUMMARY OF THE INVENTION

The present invention provides methods of producing therapeutic cells comprising providing a sample comprising hematopoietic stem or progenitor cells (HSPC) or mesenchymal stromal cells (MSC) and adsorbing a death ligand protein to a plasma membrane of the HSPCs or MSCs are provided. Therapeutic cells, and HSPCs and MSCs having adhered thereto an exogenous death ligand protein as well as compositions comprising those cells and their use in treating cancer are also provided.

According to a first aspect, there is provided a method for producing modified cells, the method comprises:
(a) providing a sample comprising hematopoietic stem or progenitor cells (HSPCs) or mesenchymal stromal cells (MSCs);
and
(b) adsorbing a death ligand protein to a plasma membrane of the HSPCs or MSCs;
thereby producing therapeutic cells.

According to another aspect, there is provided modified cells produced by a method of the invention.

According to another aspect, there is provided a hematopoietic stem or progenitor cell (HSPC) or mesenchymal stromal cell (MSC) having adhered thereto an exogenous death ligand protein.

According to another aspect, there is provided a composition comprising an HSPC, MSC or both of the invention or modified cells of the invention.

According to another aspect, there is provided a method of treating cancer in a subject in need thereof, the method comprising: administering to the subject a composition of the invention, thereby treating a cancer in a subject.

According to some embodiments, the sample comprises a population of hematopoietic cells and HSPCs are selected from the population, optionally wherein the selecting comprising selecting a subset of non-immune cells.

According to some embodiments, the sample comprising a population of hematopoietic cells is derived from umbilical cord blood (UCB), bone marrow (BM) or mobilized peripheral blood (MPB).

According to some embodiments, the sample is derived from umbilical cord blood (UCB) or bone marrow (BM).

According to some embodiments, the sample is harvested from mobilized peripheral blood by apheresis.

According to some embodiments, the MSCs are derived from bone marrow, adipose tissue, placenta or umbilical cord.

According to some embodiments, the sample is freshly harvested, preserved, or cryopreserved.

According to some embodiments, the sample is depleted of immune cells or wherein the method further comprises depleting the sample of immune cells.

According to some embodiments, the immune cells are T cells.

According to some embodiments, the sample comprises lineage-negative hematopoietic progenitors.

According to some embodiments, the sample comprises ghosts of HSPCs and MSCs, vesicles from HSPCs and MSCs, or liposomes from HSPCs and MSCs.

According to some embodiments, the death ligand protein is an exogenous death ligand protein.

According to some embodiments, the death ligand protein is a member of the tumor necrosis factor (TNF) ligand superfamily.

According to some embodiments, the TNF superfamily ligand is selected from the group consisting of Fas ligand (FasL), TNF-α, and tumor necrosis factor-related apoptosis inducing ligand (TRAIL).

According to some embodiments, the TNF superfamily ligand is FasL.

According to some embodiments, the adsorbing comprises at least one of:
a. linking the death ligand protein to the plasma membrane by an exogenous linkage;
b. providing a fusion protein comprises the death ligand protein and a binding domain that binds a component of the plasma membrane and contacting the fusion protein to the plasma membrane; and
c. providing a death ligand protein comprising a hydrophobic or lipophilic region or moiety and inserting the death ligand protein into the plasma membrane.

According to some embodiments, the exogenous linkage is a biotin-streptavidin linkage.

According to some embodiments, the method comprises non-specifically biotinylating the plasma membrane, providing the death ligand protein coupled to streptavidin and contacting the biotinylated plasma membrane with the provided death ligand protein.

According to some embodiments, the binding domain binds a protein embedded in the plasma membrane.

According to some embodiments, the binding domain binds a non-proteinaceous component of the plasma membrane.

According to some embodiments, the method comprises providing a fusion protein the death ligand protein and the hydrophobic or lipophilic region or moiety and contacting the provided fusion protein to the plasma membrane.

According to some embodiments, the death ligand protein does not comprise a transmembrane domain.

According to some embodiments, the HSPCs, MSCs or both do not comprise exogenous DNA or RNA encoding the death ligand protein.

According to some embodiments, the exogenous death ligand protein is adsorbed to a plasma membrane of the HSPC or MSC.

According to some embodiments, the exogenous death ligand protein is not imbedded in a plasma membrane of the HSPC or MSC.

According to some embodiments, the exogenous death ligand protein is not bound to its native receptor expressed from the HSPC or MSC.

According to some embodiments, the HSPC or MSC does not comprise exogenous DNA or RNA encoding the exogenous death ligand.

According to some embodiments, the exogenous death ligand:
d. is linked by an exogenous linkage to the plasma membrane;
e. is part of a fusion protein and the fusion protein comprises a binding domain that binds a component of the plasma membrane; or
f. comprises a hydrophobic or lipophilic region or moiety that is inserted into the plasma membrane.

According to some embodiments, the exogenous linkage is a biotin-streptavidin linkage, optionally wherein the plasma membrane is biotinylated and the exogenous death ligand is coupled to streptavidin.

According to some embodiments, the binding domain binds a protein embedded in the plasma membrane.

According to some embodiments, the binding domain binds a non-proteinaceous component of the plasma membrane.

According to some embodiments, the exogenous death ligand is part of a fusion protein and the fusion protein comprises the hydrophobic or lipophilic region or moiety.

According to some embodiments, the exogenous death ligand protein does not comprise a transmembrane domain.

According to some embodiments, the exogenous death ligand protein is a member of the tumor necrosis factor (TNF) ligand superfamily.

According to some embodiments, the TNF superfamily ligand is selected from the group consisting of Fas ligand (FasL), TNF-α, and tumor necrosis factor-related apoptosis inducing ligand (TRAIL).

According to some embodiments, the TNF superfamily ligand is FasL.

According to some embodiments, the composition of the invention further comprises an acceptable carrier or adjuvant.

According to some embodiments, the composition is formulated for systemic administration or intratumoral administration to a subject.

According to some embodiments, the HSPC or MSC is allogeneic, autologous or syngeneic to the subject.

According to some embodiments, the HSPC, MSC or both are extracted from the subject and the administering comprises returning the HSPCs, MSCs or both to the subject after the exogenous death ligand protein is adhered thereto.

According to some embodiments, the HSPC, MSC or both is irradiated or treated with an agent that arrest differentiation, proliferation or both before the administering.

According to some embodiments, the method further comprises administering at least one other anticancer therapy.

According to some embodiments, the at least one other anticancer therapy is selected from radiotherapy, chemotherapy and immunotherapy.

Further embodiments and the full scope of applicability of the present invention will become apparent from the detailed description given hereinafter.

However, it should be understood that the detailed description and specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

FIG. 1. Expression of FasL protein in hematopoietic progenitors has direct anti-leukemia activity.

A. BALB/c mice were inoculated with 5×10⁵ congenic A20 leukemia/lymphoma cells (H2K^{d}) and after 3 days were infused with 5×10⁶ syngeneic lineage-negative bone marrow cells (lin⁻BMC). B. Survival of recipients of naïve (n=7) and FasL-coated (n=8) lin⁻BMC.

FIG. 2. Expression of FasL protein in hematopoietic progenitors has direct and indirect anti-leukemia activity in bone marrow transplants.

A. BALB/c mice were sublethally irradiated (800 rad) and grafted with 5×10⁵ congenic A20 cells (H2K^{d}) and 5×10⁶ allogeneic (H2K^{b}→H2K^{d}) bone marrow cells (BMC). B. Survival of recipients of naïve BMC (A20+BMC, n=15) and FasL protein-coated BMC (A20+BMC-FasL, n=12). C. Viability of A20 and L1210 cells after incubation for 24 hours with oligomers of FasL protein. Data are representative of 4-5 independent incubations. D. Sublethally irradiated mice underwent allogeneic (H2K^{b}→H2K^{d}) transplants of 5×10⁶ BMC and were infused with 5×10³ L1210 congenic leukemia/lymphoma cells (H2K^{d}). Survival of recipients of naïve (BMC, n=10) and FasL protein-coated BMC (n=9).

FIG. 3. Expression of FasL protein depletes malignant cells.

A. Immunocompromised NOD.SCID mice were conditioned with two doses of 25 μg/g busulfan on days −3 and −2 and were subsequently grafted with a mixture of 3×10⁶ human Jurkat cells and equal number of human mobilized peripheral blood cells (MPB). B. Demonstrative measurements of apoptosis determined from Annexin-V uptake in gated huCD45⁺huCD3⁺ Jurkat cells in the bone marrow of recipients of naïve and FasL protein-coated T cell-depleted (TCD) MPB cells. C. Assessment of the bone marrow 3 days after infusion of 2×10⁷ human Jurkat cells (huCD45⁺huCD3⁺) showed reduced fractions in femurs of recipients of FasL protein-coated as compared to naïve lineage-negative (lin⁻) MPB. Data are representative of two femurs assessed in 5 independent experiments.

FIG. 4. Hematopoietic progenitors expressing proapoptotic proteins exert direct activity against solid tumors.

A. Murine 4T1 breast carcinoma cells (4×10⁴) were implanted subcutaneously into syngeneic mice (H2K^{d}), which were infused with 7×10⁶ syngeneic (H2K^{d}→H2K^{d}) lineage negative BMC (lin⁻BMC). Inhibition of tumor growth was more pronounced following overexpression of FasL protein (lin⁻BMC-FasL, n=9) as compared to naïve lin⁻BMC (n=6). B. BALB/c mice implanted with 5×10⁶ syngeneic (H2K^{d}) CT26 murine colon carcinoma and were grafted with naïve (BMC, n=8) and FasL protein-coated syngeneic BMC (BMC-FasL, n=9). C. Mice were inoculated subcutaneously with syngeneic (H2K^{a}) murine neuroblastoma cells (10⁶ Neuro-2a) and grafted with 7×10⁶ naïve (n=10) and FasL protein-coated (n=10) syngeneic T cell-depleted BMC (TCD-BMC). D. NOD.SCID mice with no preparative treatment were implanted with 3×10⁶ human HT29 colon carcinoma cells (day −3) and were grafted with 10⁷ naïve (n=6) and FasL protein-coated (n=8) xenogeneic human T cell-depleted mobilized peripheral blood cells (TCD-MPB).

FIG. 5. Hematopoietic progenitors expressing proapoptotic proteins reduce metastatic spread of melanoma.

A. 1.5×10⁵ B16 melanoma cells were implanted in the ear pinna of C57Bl/6 syngeneic mice (H2K^{b}) and after 14 days the visible tumors were surgically excised (control, n=11). Mice were infused intravenously with 5×10⁶ syngeneic naïve (n=10) and FasL protein-coated (n=12) lin⁻BMC and were sacrificed on day +45 for analysis of neck lymph nodes, lungs and liver. Comparative analysis of live controls (n=5) and recipients of naïve (n=5) and FasL protein-coated lin⁻BMC (n=6): B. Survival, C. Number of disease-free mice. D. Detection of surface metastases in cervical lymph nodes, lungs and liver. E. Mean number of surface metastases in the lungs and liver.

FIG. 6. Mesenchymal stromal cells overexpressing the proapoptotic ligand suppress tumor growth in vivo.

A. Demonstration of homing of allogeneic MSC (H2K^{b}→H2K^{g7}) tagged with green fluorescent protein (GFP) to the tumor stroma, as determined by surface microscopy of the 4T1 tumor (H2K^{d}) in situ in immunocompromised NOD.SCID mice. Images were acquired with an Axioplan 2 fluorescence microscope (C. Zeiss) at a magnification of 10×. B. BALB/c mice (H2K^{d}) were implanted subcutaneously with 4×10⁴ syngeneic (H2K^{d}) 4T1 cells (breast cancer) and after 3 days were infused intravenously with 10⁶ syngeneic murine MSC derived from the bone marrow (H2K^{d}). C. Tumor growth upon infusion of MSC with (n=7) and without (n=6) adsorption of FasL protein as compared to tumor growth without MSC infusion (4T1, n=7). D. Percent weight loss of the corresponding experimental groups.

FIG. 7. Direct inoculation of mesenchymal stromal cells into the bone marrow induces apoptosis in leukemic cells in vivo.

A. Mesenchymal stromal cells (2×10⁵ MSC) were inoculated into the bone marrow (IBM) of NOD.SCID mice and 2×10⁷ Jurkat cells (human T cell leukemia) were infused intravenously (IV). B. Plots represent percent human Jurkat cells (huCD45) within the contralateral femoral bone marrow of mice inoculated with naïve and FasL protein coated MSC after 3 weeks. Notably, MSC are CD45-negative and murine CD45 (mCD45) labels endogenous bone marrow cells. Data are representative of four independent experiments.

### DETAILED DESCRIPTION OF THE INVENTION

The present invention, in some embodiments provides methods of producing modified cells comprising providing a sample comprising hematopoietic stem or progenitor cells (HSPC) or mesenchymal stromal cells (MSC) and adsorbing a death ligand protein to a plasma membrane of the HSPCs or MSCs are provided. Modified cells, and HSPCs and MSCs having adhered thereto an exogenous death ligand protein as well as compositions comprising those cells and their use in treating cancer are also provided.

Hematopoietic stem and progenitor cells display inherent affinity to hematological malignancies and stroma of solid tumors, which are ideal sites of growth. As demonstrated herein, this intrinsic characteristic may be used for therapeutic purposes, essentially to target toxic factors to tumors. Considering that hematopoietic stem and progenitor cells are resistant to apoptotic signals mediated by the TNF family receptors, the cognate ligands are prime candidates for targeted delivery of anti-cancer agents. Most hematological malignancies and numerous solid tumor types are sensitive to apoptotic signaling mediated by receptor/ligand interactions of the TNF superfamily.

Provided herein, in some embodiments, a method of treating cancer, attaining targeted delivery of apoptotic signals, to malignant cells using hematopoietic progenitors as vehicles endowed with inherent affinity to tumors. The efficacy of direct anti-tumor activity of killer hematopoietic progenitors depends on the navigation capacity of the cellular vehicles and on the susceptibility of the target malignant cells to apoptosis triggered by the TNF family ligand, e.g., FasL.

By a first aspect, there is provided a method for producing cells, the method comprising:
g. providing a sample comprising cells; and
h. adsorbing a death protein to the cells;
thereby producing cells.

By another aspect, there is provided cells produced by a method of the invention.

By another aspect, there is provided a cell having adhered thereto a death protein.

By another aspect, there is provided a composition comprising the cells of the invention.

By another aspect, there is provided a method of treating a disease in a subject in need thereof, the method comprising administering to the subject the cells or composition of the invention, thereby treating a disease.

In some embodiments, the cells are modified cells. In some embodiment, the cells are therapeutic cells. In some embodiments, the cells are anticancer cells. In some embodiments, the cells are death-inducing cells. In some embodiments, the cells are proapoptotic cells. In some embodiments, are therapy is anticancer therapy. In some embodiments, the therapy is targeted therapy. In some embodiments, the therapy homes to sites of disease. In some embodiments, the disease is cancer.

In some embodiments, the sample is a blood sample. In some embodiments, the blood is peripheral blood. In some embodiments, the sample is a bone marrow sample. In some embodiments, the sample is a sample comprising cells. In some embodiments, the sample is an ex vivo sample. In some embodiments, the sample is an in vitro sample. In some embodiments, the sample is an isolated sample. In some embodiments, the sample is a purified sample. Methods of cell isolation are well known in the art, and comprise for example, Ficol separation, centrifugation, FACS cell sorting, panning, and magnetic bead coupled antibody isolation.

In some embodiments, the cells are hematopoietic stem or progenitor cells (HSPCs). In some embodiments, the cells are mesenchymal stromal cells (MSCs). In some embodiments, the cells are HSPCs or MSCs. In some embodiments, the cells are HSPCs, MSCs or both. In some embodiments, the cells are mammalian cells. In some embodiments, the mammal is human. In some embodiments, the mammal is murine. In some embodiments, the cells are not immune cells. In some embodiments, the cells are not cytotoxic cells. In some embodiments, the immune cells are immune effector cells. In some embodiments, the cells are hematopoietic cells. In some embodiments, the cells are adherent cells.

In some embodiments, the sample comprises a population of cells. In some embodiments, the sample comprises a population of hematopoietic cells and HSPCs are selected from the population. In some embodiments, the HSPCs are isolated from the population. In some embodiments, the selecting comprises excluding immune cells. In some embodiments, the selecting comprises selecting non-immune cells. In some embodiments, the selecting comprises selecting a subset of non-immune cells. In some embodiments, the selecting comprises selecting adherent cells. In some embodiments, the selecting comprises contacting the population with a death ligand and selecting cells that survive. In some embodiments, cells that survive are live cells. In some embodiments, cells that survive are non-apoptotic cells.

In some embodiments, the cells are at least 50, 60, 70, 80, 90, 95, 97, 99 or 100% pure. Each possibility represents a separate embodiment of the invention. In some embodiments, the cells are at least 70% pure. In some embodiments, the purity is purity of HSPCs or MSCs. In some embodiments, at least 50, 60, 70, 80, 90, 95, 97, 99 or 100% of the cells are HSPCs, MSCs or both. Each possibility represents a separate embodiment of the invention. In some embodiments, at least 70% of the cells are HSPCs, MSCs or both.

In some embodiments, the sample is derived from umbilical cord (UC). In some embodiments, the sample is derived from umbilical cord blood (UCB). In some embodiments, the sample is derived from bone marrow (BM). In some embodiments, the sample is bone marrow aspirate. In some embodiments, the sample is derived from peripheral blood. In some embodiments, the peripheral blood is mobilized peripheral blood (MPB). Methods of mobilizing cells from the bone marrow to migrate to the peripheral blood are well known in the art and any such method may be employed to produce the sample. In some embodiments, the sample is harvested from MPB by apheresis. In some embodiments, the sample comprises a population of hematopoietic cells derived from UCB, BM or MPB. In some embodiments, the sample comprises a population of hematopoietic cells derived from BM or MPB.

In some embodiments, the MSCs are derived from bone marrow, adipose tissue, placenta or umbilical cord. In some embodiments, the MSCs are derived from bone marrow. In some embodiments, the MSCs are derived from umbilical cord. In some embodiments, the MSCs are derived from bone marrow or umbilical cord. Methods of isolating MSCs are well known in the art and any such method may be employed. Surface markers of MSCs and non-MSC cells are well known and may be used for isolation or negative selection.

In some embodiments, the sample is freshly harvested, preserved, or cryopreserved.

In some embodiments, the sample is matched or mismatched in major histocompatibility complex antigens to a subject. In some embodiments, the sample is allogeneic to the subject. In some embodiments, the sample is autologous to the subject. In some embodiments, the sample is syngeneic to the subject.

In some embodiments, the sample is depleted of immune cells. In some embodiments, the sample is depleted of T cells. In some embodiments, the method further comprises depleting the sample of immune cells. In some embodiments, the immune cell is a cytotoxic immune cell. In some embodiments, the immune cell is an effector immune cell. In some embodiments, an effector immune cell is an immune cell with effector function. In some embodiments, the immune cell is a lymphocyte. In some embodiments, the depleting is removing. In some embodiments, the depleting comprises killing the immune cells. In some embodiments, the killing is by adding a death ligand. In some embodiments, the removing is negative selection. Methods of removing immune cells or isolating non-immune cells are well known in the art. Surface protein markers for immune cells and HSPCs are well known and may be used for isolation or negative selection.

In some embodiments, the sample comprises lineage-negative hematopoietic progenitors. In some embodiments, the sample comprises stem cells. In some embodiments, the sample is a pure sample. In some embodiments, the sample is an isolated sample. In some embodiments, the sample is an enriched sample. In some embodiments, the sample is a depleted sample. In some embodiments, the sample is a purified sample.

In some embodiments, the method comprises contacting the cell with the death ligand protein. In some embodiments, adsorbing is attaching. In some embodiments, adsorbing is adhering. In some embodiments, attaching is external attaching. In some embodiments, the adsorbing is adsorbing to a membrane of the cell. In some embodiments, the membrane is the plasma membrane. In some embodiments, the absorbing is to the cell surface. Any cell surface adsorption procedure can be used for the method of the invention. Methods of protein adsorption onto cells, are well known in the art, and include for non-limiting example insertion in the membrane (i.e., lipophilic moieties), covalent binding (i.e., fusion proteins), high affinity interactions (i.e., biotin streptavidin), specific binding to membrane constituents (i.e., glycoprotein A).

In some embodiments, the adsorbing comprises linker the death ligand protein to the plasma membrane. In some embodiments, the linkage is a chemical linkage in some embodiments, the linkage is an artificial linkage. In some embodiments, the linkage is a bond. In some embodiments, the linkage is via a linking moiety. In some embodiments, the moiety is a protein. In some embodiments, the linkage is an exogenous linkage. In some embodiments, linking comprising a linking moiety that is exogenous to the cell. In some embodiments, the linking comprising a first linking moiety on the cell surface and a second linking moiety on the death ligand protein and the linking comprising contacting the first linking moiety to the second linking moiety. In some embodiments, the first linking moiety and the second linking moiety are a binding pair. Pairs of binding molecules are well known in the art and any such pair maybe used. In some embodiments, the first linking moiety is biotin and the second linking moiety is avidin. In some embodiments, the avidin is streptavidin. In some embodiments, the linkage is a covalent linkage. In some embodiments, the linkage is a reversible linkage. In some embodiments, the linkage is an irreversible linkage.

In some embodiments, the cell is biotinylated. In some embodiments, the method comprises biotinylating the cell. In some embodiments, the method comprises biotinylating the plasma membrane. In some embodiments, the method comprises providing the death ligand protein coupled to avidin. In some embodiments, the method comprises contacting the cell with the death ligand protein. In some embodiments, the biotinylation is non-specific biotinylation. In some embodiments, the death ligand protein is biotinylated. In some embodiments, the death ligand protein is coupled to avidin. In some embodiments, the linkage is a biotin-avidin linkage.

In some embodiments, the death ligand protein is part of a fusion protein. In some embodiments, the fusion protein comprises avidin. In some embodiments, the fusion protein comprises a binding domain. In some embodiments, the binding domain binds a target exogenous to the cell. In some embodiments, the binding domain is a linking moiety. In some embodiments, the binding domain is avidin. In some embodiments, the binding domain binds a component of the plasma membrane. In some embodiments, the component is a proteinaceous component. In some embodiments, the component is an integral membrane protein. In some embodiments, the component is a surface protein. In some embodiments, the component is a receptor. In some embodiments, the component is embedded in the plasma membrane. In some embodiments, the component is a non-proteinaceous component. In some embodiments, the component is an essential membrane lipid. In some embodiments, the component is a structural component of the plasma membrane. In some embodiments, the death ligand protein is GPI anchored into the plasma membrane. In some embodiments, the fusion protein comprises a GPI-anchor. In some embodiments, the fusion protein is not GPI anchored into the plasma membrane.

In some embodiments, the death ligand protein comprises a hydrophobic region. In some embodiments, the death ligand protein is devoid of a hydrophobic region. In some embodiments, the death ligand protein comprises a lipophilic region. In some embodiments, the death ligand protein is devoid of a lipophilic region. In some embodiments, the method comprises inserting the death ligand protein into the plasma membrane. In some embodiments, the insertion is via the hydrophobic or lipophilic region. In some embodiments, the death ligand protein is not inserted into the plasma membrane. In some embodiments, the region is a transmembrane domain. In some embodiments, the death ligand protein is devoid of a transmembrane domain.

As used herein, the term “death ligand protein” refers to a protein that acts as a ligand to be bound by a surface protein, e.g., a receptor, on a target cells and whose binding induces death of the target cell. In some embodiments, the death is apoptosis. In some embodiments, the death is necrosis. In some embodiments, the death ligand protein is a proapoptotic protein. In some embodiments, the death ligand protein is an exogenous death ligand protein. In some embodiments, the death ligand protein is not expressed by the cells. In some embodiments, the death ligand protein is not naturally expressed by the cells. In some embodiments, the cells do not comprise exogenous nucleic acid molecules that encode the death ligand protein. In some embodiments, the nucleic acid molecule is DNA. In some embodiments, the nucleic acid molecule is RNA.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the death ligand protein is a member of the tumor necrosis factor (TNF) ligand superfamily. The TNF family of receptors is well known in the art, as are their ligands. Ligands of this superfamily that induce cell death are also known and the amino acids sequences of these molecules as well as nucleotide sequences encoding them are readily available online and well known to the skilled artisan. In some embodiments, the TNF superfamily ligand is selected from the group consisting of Fas ligand (FasL), TNF-α, and tumor necrosis factor-related apoptosis inducing ligand (TRAIL). In some embodiments, the TNF superfamily ligand is FasL. TNF superfamily ligand is TNF-α. In some embodiments, the TNF superfamily ligand is TRAIL. In some embodiments, the TNF superfamily ligand is not TRAIL. It will be understood by the skilled artisan that any suitable death-inducing molecule may be employed.

As described herein, the current application, in some embodiments thereof, provides to substitute immune cells with hematopoietic progenitors for targeted delivery of death signals to kill apoptosis-sensitive hematological malignancies and solid tumors (hereinafter: “apotherapy”).

Hematopoietic progenitors may be advantageous for use as vehicles for delivery of apoptotic signals, for several reasons. First, hematopoietic progenitors are resistant to apoptotic signaling, and therefore can carry lethal TNF family ligands to tumors without being affected. Insensitivity of the hematopoietic progenitors to radiochemotherapy indicates that apotherapy may be intercalated as an adjuvant in between cycles of conventional therapy. Furthermore, these cellular vehicles are insensitive to prevalent tumor-defense mechanisms and overcome limitations of immune surveillance. Second, hematopoietic progenitors are endowed with inherent affinity to sites of growth such as tumor stroma, and therefore navigate to detect small tumoral implants. In fact, these cells use similar mechanisms of chemotaxis and adhesion to home to the bone marrow and to migrate to tumor stroma. Third, homing to the bone marrow space attacks the most prevalent common hideout of residual disease in hematological malignancies as well as a series of solid tumors. Approximately 10% of hematopoietic progenitors, best defined as lineage-negative bone marrow cells (lin⁻BMC), home to the bone marrow. Fourth, hematopoietic progenitors are trapped in the most prevalent tissues hosting metastatic spread. Approximately 70% of intravenously infused cells are retained by passive filtration in the lungs and liver, which are the main sites of hematogenic metastasis, in addition to incorporation in the reticuloendothelial system that represents the major route of lymphatic metastatic spread. Fifth, apotherapy targets tumor stroma and therefore indiscriminately kills cancer cells including slow cycling tumor initiating cells. Radiochemotherapy affects primarily fast cycling cells, immunotherapy recognizes tumors according to their immunogenic configuration and can eliminate mitotically quiescent and slow-cycling cells. Finally, autologous cells are not rejected, expression of ectopic proteins avoids introduction of genetic materials, and hematopoietic progenitors thrive under signals of the TNF superfamily, thus fostering recovery from hypoplasia after radiochemotherapy. The low toxicity of apotherapy makes it a useful anti-cancer tool after hematopoietic cell transplants associated with extended periods of weeks to months of immunosuppression and absent effective immune surveillance of tumors.

As described herein below, the killing capacity of hematopoietic progenitors was empowered by means of transient overexpression of proteins encoding TNF family ligands. In a series of murine and surrogate human experiments the inventor demonstrates several major activities of hematopoietic progenitors overexpressing a streptavidin and Fas-ligand chimeric protein, which is easily adsorbed onto cell surface via biotinylation. These cell-protein conjugates exert potent direct anti-tumor activity against murine and human leukemia/lymphoma cells and colon carcinoma, and murine solid tumors including neuroblastoma and breast carcinoma. Major advantages include the quite unique intrinsic affinity of hematopoietic progenitors to the primary sites of tumor metastasis (lungs, liver and reticuloendothelial system) and particular homing to the bone marrow, their natural site of residence that also serves as the most prevalent hideout of residual disease that causes relapse of hematological malignancies and solid tumors. Furthermore, resistance to apoptosis disarms tumor mechanisms of defense from immune surveillance through induction of apoptosis in immune cells, and may be implemented in conjunction with standard radiochemotherapy, immunotherapy and bone marrow transplantation.

The therapeutic efficacy of the proposed approach has been documented in a number of experiments showing direct killing of malignant cells. One experiment demonstrates rescue of mice by killer hematopoietic progenitors from death caused by leukemia/lymphoma of murine (Examples 1,2) and human origin (Example 3). Another experiment presents direct effects of murine killer hematopoietic progenitors on prevalent solid tumors including murine neuroblastoma, breast and colon carcinoma (Example 4). All these experiments document isolated activity of autologous killer hematopoietic progenitors without any preparatory regimen.

In some experiments, inhibition was assessed under conditions of uncontrolled growth of tumors in immunosuppressed mice. One embodiment exhibits recipients conditioned by total body irradiation, to simulate immunosuppression that frequently follows radiochemotherapy and transplantation. In this model, allogeneic hematopoietic cell engraftment rescued 20% of the mice, whereas infusion of killer hematopoietic progenitors rescued of ˜75% of the mice, even under uncontrolled expansion of the malignant cells (Example 2). Another model involved implantation of malignant cells in immunocompromised NOD.SCID mice. Infusion of T cell-depleted mobilized peripheral blood cells expressing FasL protein reduced the burden of human leukemia (Example 3) and colon carcinoma (Example 4). Direct toxicity was further emphasized in these models, as tumor growth inhibition preceded by far the temporal frame of murine and human cell engraftment in allogeneic and xenogeneic recipients, respectively.

Direct activity of killer cells was also documented using mesenchymal stromal cells as vehicles in murine breast carcinoma (Example 6) and hematological malignancies such as human leukemia/lymphoma (Example 7). Mesenchymal cells migrate with good affinity to the stroma of solid tumors, however, do not home efficiently to the bone marrow. Therefore, in one example therapeutic MSC were infused directly into the bone marrow, emphasizing fractional reduction in malignant cell burden in the contralateral femur (Example 7) as observed with efficient bone marrow homing of killer hematopoietic progenitors infused intravenously (Example 3). The particular migration patterns of mesenchymal cells should be considered for their therapeutic implementation as vehicles for apoptotic signals.

The unequivocal mechanism of action of killer hematopoietic progenitors and mesenchymal stromal cells was reduction of the burden of malignant cells through apoptotic signaling by FasL protein. Increased fractional death through apoptosis of malignant cells within the bone marrow early after infusion of therapeutic cells (Example 3) was directly related to increased survival (Examples 1,2). Similarly, direct inoculation of killer mesenchymal stromal cells into the bone marrow reduced the fraction of human Jurkat leukemia/lymphoma (Example 7).

Efficient anti-cancer activity was attained by lineage-negative hematopoietic progenitors derived from the murine bone marrow (Examples 1,2,4,5) and human mobilized peripheral blood (Examples 3,4). Progenitors negative for lineage markers generally consist of ˜5% of total bone marrow cellularity, similar numbers of progenitors may be collected by apheresis from peripheral following mobilization. For practical purposes, the bone marrow is a virtually unlimited source of progenitors that replenishes itself at high rates and may be enriched with precursors by selective depletion of differentiated cells using agents such as fluorouracil. Likewise, progenitors may be repeatedly mobilized into peripheral blood without any impact on the proper hematopoietic function of the marrow compartment. As demonstrated herein, mobilized peripheral blood depleted of T cells and lineage-positive cells are effective vehicles for targeted therapy (Examples 3,4). An additional source of progenitors is umbilical cord blood, which may be either procured at birth for further autologous use or as a source of readily available cells for allogeneic cell therapy. Possible ex vivo expansion of the progenitors is not excluded to attain large numbers of cells for therapeutic implementation.

Autologous bone marrow cells may be used as bulk preparations; however, selection of lineage-negative progenitors or depletion of T cells may improve safety (Examples 1-5). On the one hand, navigation of hematopoietic progenitors to tumors is much more efficient that other differentiated cells. On the other hand, overexpression of the apoptotic ligand in progenitors is sufficient for the therapeutic effect, whereas other cell subsets might cause unnecessary side effects. Importantly, hematopoietic stem and progenitor cells are resistant to apoptotic signaling transduced by TNF family receptor/ligand interactions.

Autologous progenitors are best implemented without rejection; thus, the therapeutic window may be limited by the lifetime of chimeric proteins adsorbed onto cell surface. The streptavidin-FasL chimeric protein is expressed on cells via the high affinity streptavidin-biotin interaction and has a half lifetime of 3.5-4 days on the surface of cells in vivo. Hematopoietic progenitors cycle at slow rates at extramedullary sites and only ˜25% of bone marrow-homed cells proliferate within the effective therapeutic time frame. Mesenchymal stromal cells are slow cycling and are unlikely to proliferate significantly within the therapeutic period of 7-10 days.

Allogeneic hematopoietic progenitors may be used within the time frame of apotherapy, as overexpression of the ectopic protein defends these cells from immune rejection. The killer cells actively counter rejection by presentation of death signals to alloreactive effectors in immunocompetent recipients, thus making possible the use of allogeneic hematopoietic progenitors within the therapeutic interval of this approach. Also contemplated is the use of universal sources of hematopoietic progenitors and mesenchymal stromal cells, such as placenta and umbilical cord, as well as ex vivo expansion of these progenitors.

A common denominator of adoptive transfer of any cell type is trapping by passive filtration in the liver and lungs. It is estimated that approximately 60-70% of the infused cells are retained in these compartments through mechanical filtration. This characteristic is of outmost importance for primary tumors in these organs such as the prevalent lung cancer and hepatocellular or cholangic carcinomas, which are rather poorly responsive to known radiochemotherapy. Furthermore, the lungs and liver are the most prevalent sites of metastasis of most cancers, including leukemia/lymphoma cells (Examples 1,2) and melanoma (Example 5) used in our models. Direct effect on metastatic tumoral implants was best demonstrated by the significant relief from iatrogenic spread of melanoma metastases in the lungs, liver and lymph nodes attained by infusion of killer hematopoietic progenitors. Therapy increased two-fold short-term survival, with no evidence of disease in ˜40% of the mice.

Hematopoietic progenitors are also effective in targeting the reticuloendothelial system, which is one of the major sites of metastasis of numerous hematological and non-hematological cancers (Example 5). This phenomenon is well known from the initial and rather ineffective extramedullary hematopoietic colonies found in the spleen following hematopoietic cell transplants.

Hematopoietic stem and progenitor cells home with outmost efficacy to the bone marrow, their physiological site of residence and activity. It has been estimated that approximately 10-15% of lineage-negative progenitors home to the bone marrow of conditioned and non-conditioned recipients. Current data demonstrate efficient homing and delivery of apoptotic signals within the bone marrow to reduce the burden of malignant cells (Example 3), which is significant for several reasons. First, primary hematological malignancies such as leukemia, lymphoma and multiple myeloma use bone marrow niches for excessive growth, a compartment efficiently targeted by selective homing of hematopoietic progenitors. Second, bone is one of the major sites of metastasis of numerous cancers, including hematological malignancies and solid tumors such as breast and prostate carcinoma. Third, primary osteosarcomas evolving in the bone such as Ewing sarcoma, are rather poorly responsive to radiochemotherapy but would be effectively targeted by killer hematopoietic progenitors. Fourth, the marrow space is one of the prevalent hideouts of malignant cells responsible for relapse following aggressive radiochemotherapy, not only of hematological malignancies such as leukemia and lymphoma but also solid tumors such as neuroblastoma.

Hematopoietic progenitors and mesenchymal stromal cells have the fundamental capacity to cross barriers and infiltrate immune privileged organs such as the eye and brain, compartments that host and shield evolution of malignant tumors such as ocular melanoma and brain glioblastomas. These cells are therefore effective vehicles due to inherent capacity to cross the blood-brain barrier and membranal barriers of vital and reproductive organs, malignant cell hideouts responsible for disease relapse.

Killer hematopoietic progenitors have access to and operate within immune privileged sites, which are largely inaccessible to the immune system. Like physiological immune privilege, tumors employ TNF family ligands and other negative regulators of the immune system, such as Cytotoxic T lymphocyte antigen-4 and Programed death-1 ligand, to counterattack the activity of immune cells reactive against tumoral antigens. Unlike immune cells, the use of apoptosis-resistant hematopoietic progenitors as vehicles disarms this mechanism of tumor defense from immune surveillance and abolishes tumoral immune privilege.

Most hematological malignancies and prevalent solid tumors are sensitive to apoptotic signaling, whereas resistance to this mechanism is often associated with undifferentiated states, primitive phenotypes and aggressive behavior. Evaluation of a less sensitive leukemia/lymphoma cell line showed significant effects and rescue from death of approximately 75% of the mice (Example 2), further documenting the efficacy of the proposed therapeutic approach to tumors with moderate sensitivity to apoptotic signaling.

Inherent affinity of hematopoietic progenitors and mesenchymal stromal cells to tumor stroma is mediated by directed migration to sites that offer favorable environments for engraftment and growth. These cells might incorporate and foster formation of tumor stroma, for example by contribution of chemokines and cellular elements to vascularization of the tumor. Overexpression of death molecules on the surface of the therapeutic cells prevents such incorporation in the tumor stroma, because neighboring cellular stromal elements are rather sensitive to apoptotic signals.

This application further provides an evolution of cancer immunotherapy, where activated immune cells are substituted by hematopoietic progenitors and transient killing capacity is awarded through adsorption of chimeric proteins to attain effective anti-tumor activity. This evolution avoids non-selective systemic immune stimulation required to attain maximal activity of autologous and allogeneic immune cells against cancer. The cellular vehicles target tumor stroma, do not incorporate but abolish one of the main mechanisms of tumor evasion from immune surveillance through presentation of apoptotic signals to reactive immune cells. Thus, the invention provides a method which turns the tumor defense strategy against the malignant cells themselves.

The biology of FasL is complex, with initial presentation as a membrane-bound molecule that is the common executioner of apoptosis within the TNF family. The membrane bound FasL isoform awards tumors the configuration of immune privilege and counterattack immune surveillance by physical elimination of tumor infiltrating lymphocytes. Shedding of soluble FasL from cell surface by matrix metalloproteinases is accompanied by loss of the apoptotic activity through trimerization of the Fas receptor. Soluble FasL has diverse activities that promote inflammatory environments and enhance metastatic spread by activation of migration pathways. As provided herein, the use of non-cleavable FasL protein overcomes this drawback. In some embodiments, the FasL is non-cleavable FasL.

Multiple possible applications are envisioned in the context of apoptotic therapy to cancer by non-immune cells overexpressing apoptotic ligands. This approach to cancer debulking may be used in the induction phase along conventional radiochemotherapy and immunotherapy, may be used as an adjuvant in the consolidation phase of therapy or may be applied alone either for eradication of minimal residual disease or as a palliative measure in metastatic disease. The therapeutic effect obviates the need for complex bone marrow transplant procedures and is devoid of graft versus host reactivity. Therefore, it can be repeatedly applied as adoptive apotherapy to recipients under transient immunosuppression, which is associated with considerably less toxicity than the mildest regimens of non-myeloablative and reduced intensity conditioning.

Insensitivity of hematopoietic progenitors to apoptosis and radiochemotherapy awards apotherapy a prime role in complementing current therapies for cancer. One possible implementation of killer hematopoietic progenitors suggests intercalation in between standard radio-chemotherapeutic procedures, as the vehicles are generally resistant to radiation and chemical agents. Furthermore, the use of apotherapy as an adjuvant has three significant advantages. First, radio-chemotherapy induces Fas receptor expression, which is one of the pivotal mechanisms of tumor cell death, similar to execution of apoptosis in immunotherapy. For example, Fas is induced and upregulated by fluorouracil in colon and hepatocellular cancer, by gemcitabine in lung cancer, by doxorubicin and cisplatin in neuroblastoma, by taxol in prostate and breast cancer, similar to irradiation and histone deacetylase inhibitors. Second, transition to adaptive immunity by immunosuppression is often beneficial to generation of anti-tumor reactions by patient's own immune system. All chemotherapeutic agents, targeting fast cycling cells, have immunosuppressive activity at some extent. Resetting the immune system stands at the basis of autologous immune-hematopoietic reconstitution following aggressive radiochemotherapy (often termed autologous bone marrow transplants). Third, TNF family ligands transduce trophic signals in progenitors that augment engraftment and foster immune-hematopoietic reconstitution, shortening the period of hypoplasia following radiochemotherapy and transplantation.

Apotherapy may substitute GvT reactions prior to cancer immunization by transition to either autologous or allogeneic adaptive immunity following bone marrow transplantation. All transplant procedures, autologous, allogeneic and haploidentical, are associated with periods of weeks to months during which there is no effective immune surveillance of the tumors prior to functional immune reconstitution.

Targeted apotherapy may precede immunotherapy in order to inflict local injury to tumors and foster antigen uptake and presentation and consequently foster immune sensitization. Therefore, the proposed approach may be used to increase cancer immunogenicity by induction of apoptosis in cells at the tumor perimeter, as often attained by low doses of radiochemotherapy. Subsequently, immune cells endowed with endogenous or engineered receptors recognize tumor antigenic epitopes and initiate immune reactions against the malignant targets. Likewise, it may follow immunotherapy in order to prevent cancer escape from antigen recognition, often attributed to active defense, genomic instability and immunoediting. One may use allogeneic hematopoietic progenitors to elicit immune reactivity, a general reaction that fosters anti-tumor responses through ample supply of cytokines and activation of various cell types.

Expression of the ectopic TNF family ligands awards hematopoietic progenitors a status of immune privilege through direct elimination of residual host immune cells that mediate rejection. Thus, allogeneic and haploidentical hematopoietic progenitors may be used for apotherapy as “off the shelf” preparations. Even in the case of rejection, the time frame is within the therapeutic window of efficient apotherapy of 7-10 days, attributed primarily to shedding of the ectopic proteins from the cells surface. Repeated infusions of immune privileged allogeneic and haploidentical hematopoietic progenitors under conditions of minimal cytoreductive conditioning may result in progressive evolution of stable mixed hematopoietic chimerism, endowed with the capacity to elicit sustained GvT reactions while obviating the complex hematopoietic cell transplant procedure.

These general potential applications to cancer therapy benefit of several distinct advantages, which evolve primarily from the inherent characteristics of the two components: hematopoietic progenitors serving as the targeting moiety and TNF family ligands acting as the killing moiety. Hematopoietic progenitors and mesenchymal stromal cells resistant to apoptotic signaling migrate with high affinity to tumors and simulate the physiological mechanism of cancer cell eradication by TNF family signaling.

First, targeted delivery of apoptotic signals using vehicles with inherent affinity to tumors is a distinct therapeutic approach that shares characteristics with other therapeutic modalities. Like radiochemotherapy, malignant cells undergo apoptotic death in response to activation of the TNF family receptors. Like immunotherapy, apoptotic signals are delivered independent of the pace of cell cycling and therefore this approach is effective against tumor initiating cells that display outstanding resilience to radiochemotherapy. Apotherapy targets tumor stroma and enforces cancer cell death irrespective of the cycling rate of the malignant cells, whereas almost all chemotherapeutic agents and irradiation target exclusively fast cycling cancer cells.

Second, hematopoietic progenitors migrate with high inherent affinity to the prevalent sites of metastasis. The physiological mode of hematopoietic progenitor distribution includes passive trapping through filtration in the lungs and liver, retention in the reticuloendothelial system (including spleen and lymph nodes) and active homing to the bone marrow, the physiological site of residence. Lungs, liver, lymph nodes and bones are targeted as the most prevalent sites of metastasis of most cancers that spread through hematogenic and lymphatic routes of dissemination.

Third, the proposed approach to cancer effectively targets immune privileged sites and is optimal for attack of hideouts for minimal residual disease. The bone marrow is a relatively immune privileged compartment, which hosts and shields numerous hematological malignancies (lymphoma, leukemia, multiple myeloma) and solid tumors (neuroblastoma). The vehicles also penetrate other immune privileged sites such as the brain and reproductive organs that serve as prevalent hideouts for malignant cells, both because are inaccessible to and actively disable immune cells.

Fourth, apoptosis-resistant hematopoietic progenitors overcome defense mechanisms of tumors and their stroma, whereas apoptosis-sensitive immune cells are eliminated in tumor microenvironment without causing injury. Tumors create relative immune privilege by expression of TNF family ligands among other agents that defend from immune attack by induction of apoptosis in immune cells. intrinsic insensitivity of hematopoietic progenitors and mesenchymal stromal cells to apoptosis make them optimal vehicles for targeted delivery of apoptotic signals and death molecules.

Fifth, the bone marrow is an unlimited source of progenitors, which can be directly harvested or mobilized and collected form peripheral blood. Thus, the procedure may be repeatedly applied by frequent infusion of large numbers of therapeutic cells at close intervals. Furthermore, repeated infusions of allogeneic hematopoietic progenitors immune privileged by means of cell surface expression of apoptotic ligands may lead to stable engraftment and generation of sustained endogenous anti-tumor reactions generated by the allogeneic immune cells, while minimizing the threat of graft versus host disease that is not elicited by the host-tolerant alloimmune progeny.

Finally, the proposed approach alleviates some of the prevalent side effects of other therapeutic modalities: a) The proposed approach to cancer therapy avoids introduction of ectopic genetic material and engineered cells, and uses proteins with limited lifetime; b) Allogeneic hematopoietic progenitors and the evolving immune progeny are devoid of graft versus host reactivity, alleviating one of the threats associated with hematopoietic cell transplants and stable engraftment of allogeneic T cells; c) Evidently there is no need for immune stimulation, the systemic consequences of which pose most severe difficulties in implementation of humoral and cellular immunotherapy to cancer.

Toxicity of TNF family ligands has been intensively investigated. Initial hepatic toxicity reports of an activating Fas antibody (Jo2) which was later shown to originate from the Fc terminal, and numerous studies showed that the Fas/FasL interaction is devoid of toxicity generated by the particular antibody. Likewise, induced expression of TRAIL has been found in various studies to have no significant side effects in vivo. TNFα has been widely used to treat local malignancies in the extremities and the threshold levels for systemic toxicity are well known.

Limits of safety for use of TNF family ligands have been set in the clinical setting. TNF-α has been used several decades for systemic therapy at two daily doses of 200 μg/m² (4.6 nmol/ml) and for treatment of malignancies such as melanoma using isolated limb perfusion of the extremities at doses of 4-6 mg TNFα. In isolated limb preparations, recorded serum levels varied widely from 14 (80 nmol/ml) to 277 ng/ml, representing toxic non-lethal doses, that might cause fever, nausea/vomiting, tachycardia, hypotension and rise in bilirubin. Both prevalent and infrequent toxicities were transient and responded well to supportive treatment. Likewise, the toxic doses of biotin, proposed to be used as one of the techniques to anchor proteins to the cell surface, are estimated at 1 mg/Kg (equivalent to 0.65 nmol/ml).

Cells expressing saturating amounts of streptavidin via botinylation retain approximately 1.4×10⁵ chimeric molecules per cell. Administration of 5×10⁸ therapeutic hematopoietic cells/Kg to an 80 Kg recipient requires infusion of a total of 4×10¹⁰ cells. Expression of the fusion proteins via biotinylation at absolute efficiency results in a total amount of approximately 330 μg FasL and 440 μg TNFα (0.002 nmol/ml) ectopic protein expressed on the cell surface. This amount represents concentrations of the ligands 3 orders of magnitude lower than the intravenous dose, 4.5 orders of magnitude lower than the minimal reported toxic concentrations for the apoptotic ligands in isolated limb perfusion, and 3.5 orders of magnitude lower than the toxic levels of biotin. These are rather inaccurate maximal levels of the two components of chimeric proteins, which are cleaved from the cell surface with a half-life time of ˜3.5 days in vivo, within tissues and are not directly released into systemic circulation.

While the goal is to take advantage of the particular inherent characteristics of the vehicles and the target cells, further modifications may be performed to overcome relative limitations: a) Increase the susceptibility to apoptosis of the target tumor cells through: i. enhanced expression of TNF family receptors using therapeutic or reduced doses of radiochemotherapy, and ii. cross talk between TNFα family receptors, such as minute amounts of TNFα prior to administration of progenitors coated with FasL protein. b) Reinforce the navigation capacity of hematopoietic progenitors and mesenchymal stromal cells to tumors by increasing expression of adhesion molecules. Concerns of potential detrimental contribution of hematopoietic progenitors and particularly of mesenchymal stromal cells to tumor growth and stroma may be removed by irradiation of the therapeutic cells. Disruption of DNA by radiation prevents cell proliferation and abolishes the synthetic activity. Abrogation of the differentiation capacity of cells used as therapeutic vehicles will, for example, preclude possible contribution to tumor vascularization and incorporation in tumor stroma. In addition, the therapeutic vehicles are unable to secrete growth and supportive factors that might foster cancer cell growth. d) Membranal structures such as ghosts of hematopoietic progenitors and mesenchymal stromal cells can be used for targeted delivery of toxic moieties. Considering that chemotactic and adhesion molecules are expressed on the surface of cell membrane, physiological migration and homing of the ghosts will be similar to that of live cells. Therefore, it is conceived possible use of vesicles, liposomes and nanoparticles for direct delivery of TNF family ligands to sites of interest.

In some embodiments, the composition comprises the cells of the invention. In some embodiments, the composition comprises a therapeutically effective amount of the cells. In some embodiments, the composition comprises the modified cells of the invention. In some embodiments, the composition comprises the HSPCs of the invention. In some embodiments, the composition comprises the MSCs of the invention. In some embodiments, the composition is pharmaceutical composition. In some embodiments, the composition is a therapeutic composition. In some embodiments, the composition comprises an acceptable carrier or adjuvant. In some embodiments, acceptable is pharmaceutically acceptable.

As used herein, the term “carrier,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the compositions presented herein.

In some embodiments, the composition is formulated for systemic administration. In some embodiments, the composition is formulated for administration to a subject. In some embodiments, the composition is formulated for intratumoral administration. In some embodiments, the composition is formulated for intravenous administration. In some embodiments, the cells are allogenic to the subject. In some embodiments, the cells are autologous to the subject. In some embodiments, the cells are syngeneic to the subject.

In some embodiments, the disease is cancer. In some embodiments, the method is a method of treating cancer. In some embodiments, the disease is a proliferative disease. In some embodiments, the method is a method of treating a proliferative disease. In some embodiments, proliferative is hyperproliferative. In some embodiments, the cancer is any cancer. In some embodiments, the cancer is a solid cancer. In some embodiments, the cancer is a hematopoietic malignancy. In some embodiments, the cancer is a tumor. In some embodiments, the cancer is a hematological cancer. Non-limiting examples of cancer which may be treated by the method provided herein include head and neck cancer, melanoma, ovarian cancer, prostate cancer, cervical cancer, renal-cell carcinoma, hepatic colorectal carcinoma, colorectal cancer, hepatocellular carcinoma, liver cancer, lymphoma, leukemia and metastases thereof. In some embodiments, the cancer is an immune cell cancer. In some embodiments, the cancer is a B cell cancer. In some embodiments, the cancer is a T cell cancer. In some embodiments, the cancer is a lymphoma. In some embodiments, the cancer is a leukemia.

In some embodiments, the cells are extracted from the subject and the administering comprises returning the cells to the subject after the death ligand protein is adsorbed thereto. In some embodiments, the cells are treated before administration. In some embodiments, the treating is a treating that inhibits proliferation, differentiation or both. In some embodiments, inhibits is abolishes. In some embodiments, inhibits is arrests. In some embodiments, the treating is irradiating. In some embodiments, the treating is contacting with an agent that inhibits proliferation, differentiation or both.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intratumoral, intramuscular, or intraperitoneal.

The dosage administered will be dependent upon the age, health, and weight of the recipient, kind of concurrent treatment, if any, frequency of treatment, and the nature of the effect desired.

In some embodiments, the method of the invention further comprises administering another therapy. In some embodiments, at least one other therapy is administered. In some embodiments, the therapy is an anticancer therapy. In some embodiments, the anticancer therapy is selected from radiotherapy, chemotherapy and immunotherapy. In some embodiments, the anticancer therapy is radiotherapy. In some embodiments, the anticancer therapy is chemotherapy. In some embodiments, the anticancer therapy is immunotherapy. One skilled in the art will appreciate that the standard care for a disease will depend on many factors such as the type of disease, the stage/severity of the disease, the age and health of the patients and other information as may be available to the physician at the time of treatment.

The term “therapeutically effective amount” refers to an amount of a composition effective to treat a disease or disorder in a mammal. In some embodiments, a therapeutically effective amount is an amount effective, at dosages and for periods of time necessary, to achieve the desired therapeutic result. The exact dosage form and regimen would be determined by the physician according to the patient's condition. In some embodiments, the dosage is a repeat dosage.

The doses can be single doses or multiple doses over a period of several days, weeks, months or even years or for as long as it is beneficial to the subject. The treatment generally has a length proportional to the length of the disease process and treatment effectiveness and the patient species being treated.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994 Mishell and Shiigi (eds)), “Strategies for Protein Purification and Characterization—A Laboratory Course Manual” CSHL Press (1996); all of which are incorporated by reference. Other general references are provided throughout this document.

### EMBODIMENTS

### Embodiment 1. Progenitors Expressing Death Ligands Induce Apoptosis in Malignant Cells

### Example 1: Expression of Fas-Ligand in Hematopoietic Progenitors Exerts Direct Toxicity to Apoptosis-Sensitive Leukemia Cells In Vivo

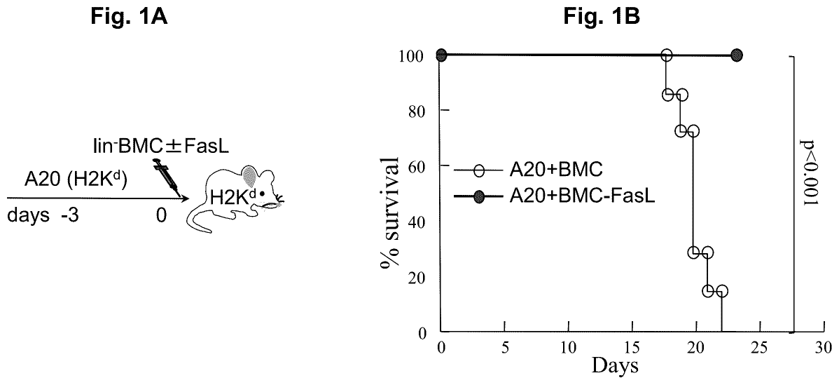

Most hematological malignancies are sensitive to apoptosis. We used a model of A20 murine leukemia/lymphoma cells to test the therapeutic efficacy lineage-negative hematopoietic progenitors (lin⁻BMC) overexpressing an ectopic FasL protein without any preparatory conditioning (FIG. 1A). Joint infusion of congenic A20 cells and syngeneic lin⁻BMC overexpressing FasL protein rescued all mice, whereas infusion of naïve hematopoietic progenitors resulted in death of all mice within 23 days (FIG. 1B). The main cause of death was widespread dissemination of the malignant cells to the liver and lungs. These data emphasize potent direct anti-leukemia activity following expression of the proapoptotic ligand in hematopoietic precursors.

### Example 2: Hematopoietic Progenitors Expressing Ectopic Fas-Ligand Exert Anti-Leukemia Activity in Immunosuppressed Mice In Vivo

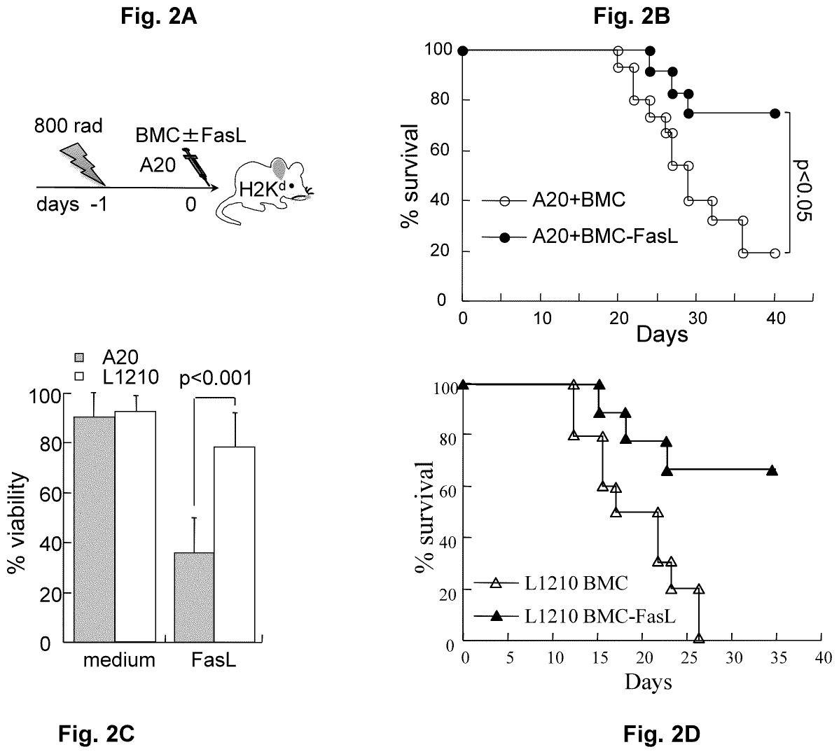

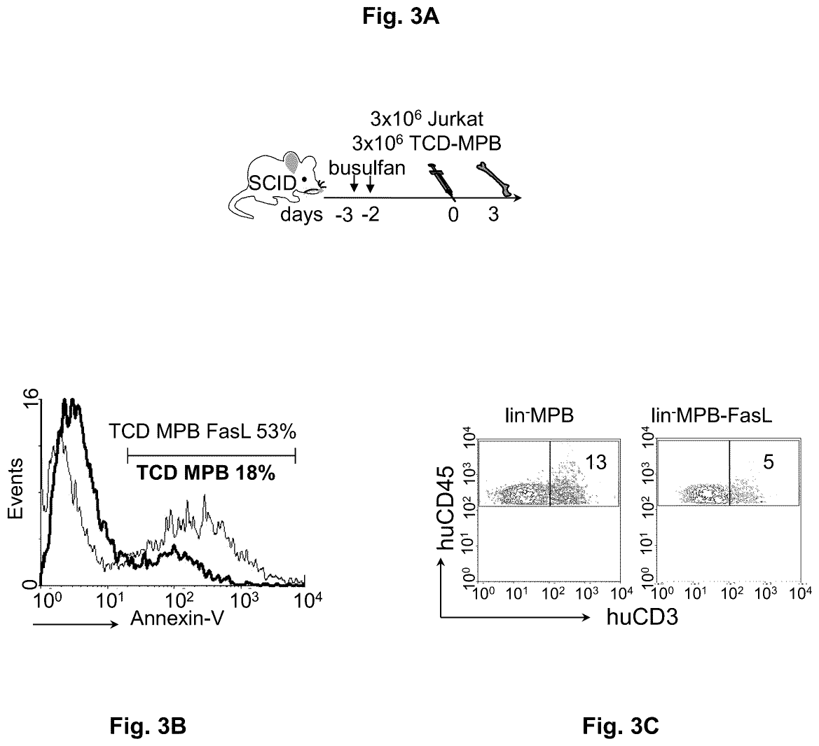

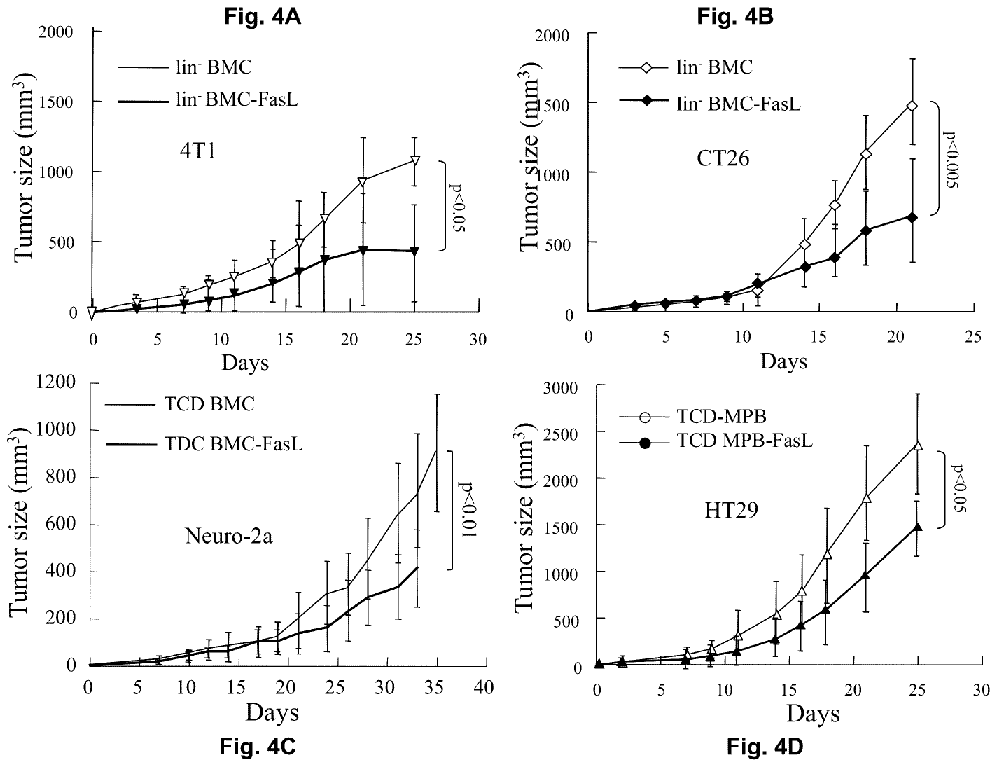

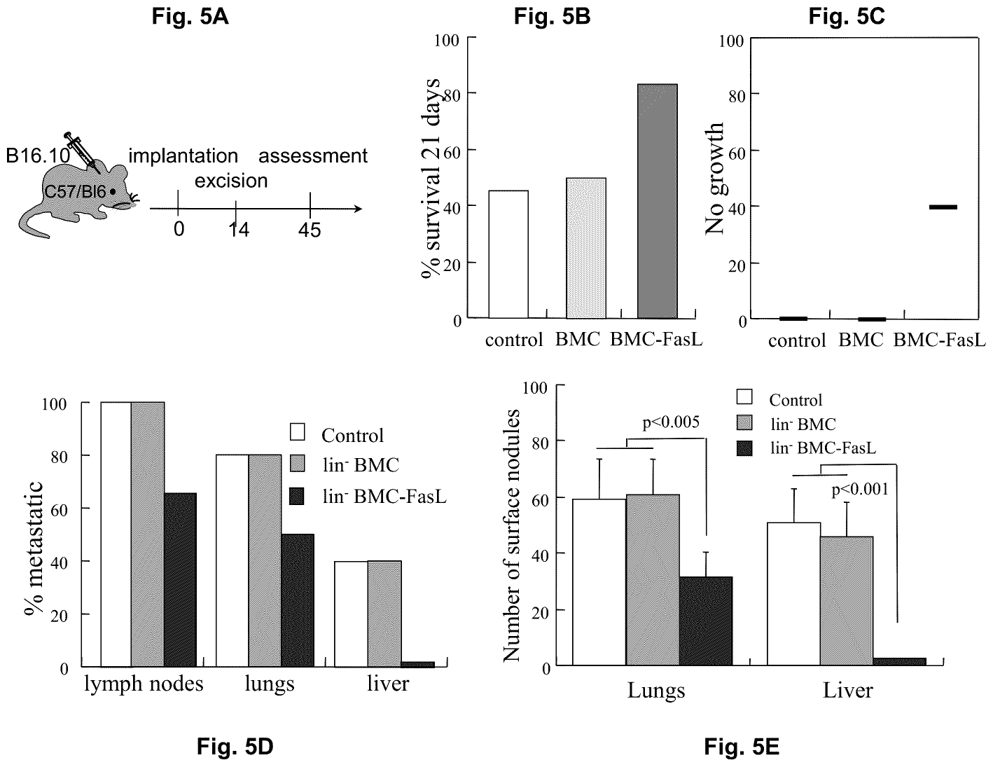

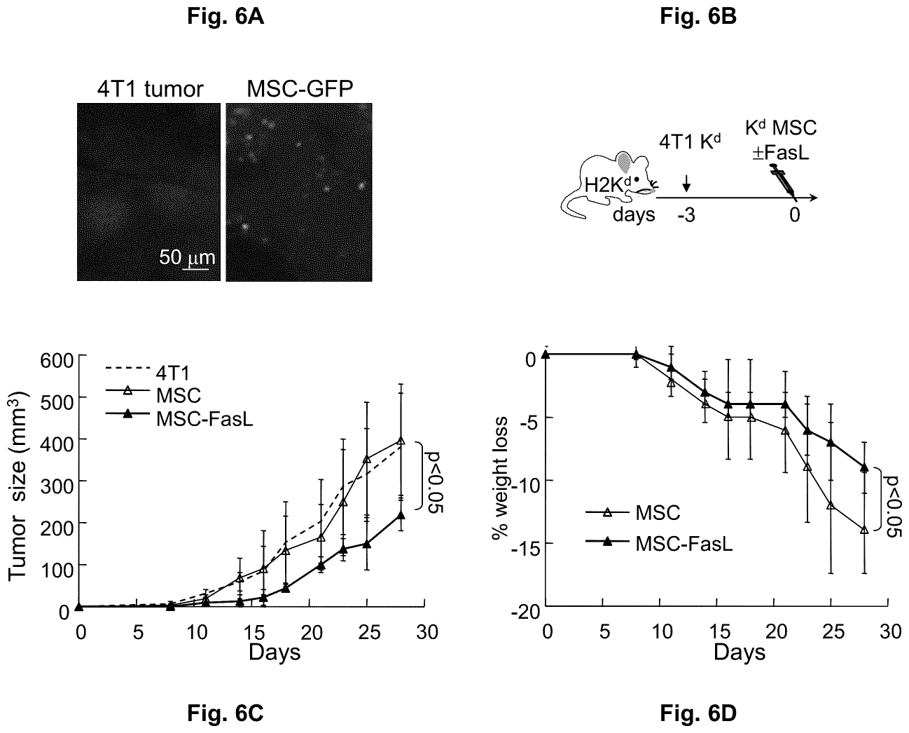

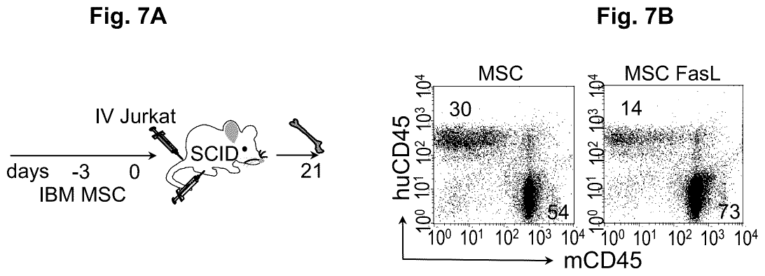

To simulate conditions of immunosuppression, such as after administration of radiochemotherapy and conditioning for bone marrow transplantation, mice were sublethally irradiated prior to infusion of A20 cells and allogeneic (H2K^{b}→H2K^{d}) BMC (FIG. 2A). Although engraftment of naïve allogeneic BMC rescued ˜20% (3/15) of the recipients (FIG. 2B), grafting of bone marrow cells overexpressing ectopic FasL protein rescued 75% (9/12) of the recipients (p<0.05). Robust activity of the therapeutic cells is documented under conditions of uncontrolled tumor growth in immunosuppressed mice.

To evaluate a less sensitive cell line, L1210 murine leukemia/lymphoma cells were assayed for direct Fas-mediated apoptosis in vitro (FIG. 2C). Using the same experimental model (FIG. 2A), all sublethally irradiated recipients of naïve BMC succumbed to death within 26 days, whereas 66% (6/9) recipients of FasL-coated BMC survived (FIG. 2D). The therapeutic killer cells display significant activity against hematological malignancies with intermediate sensitivity to Fas-mediated apoptosis.

### Example 3: Human Hematopoietic Progenitors Expressing Ectopic Fas-Ligand Induce Apoptosis in Human Leukemia Cells in the Bone Marrow In Vivo

To evaluate the mechanism of protection mediated by killer hematopoietic progenitors, a series of studies was performed to detect apoptosis of the malignant cells in mice. Immunocompromised NOD.SCID mice were prepared with two doses of busulfan, a cytoreductive agent used to increase homing of the malignant and therapeutic cells to the bone marrow. Mice were infused with Jurkat human leukemia/lymphoma B cells and human T cell-depleted mobilized peripheral cells (TCD-MPB) (FIG. 3A). The bone marrow of NOD.SCID recipients was evaluated after 3 days for detection of human Jurkat cells, identified as huCD45⁺huCD3⁺ and apoptosis as determined from Annexin-V incorporation. Apoptosis of Jurkat cells was markedly increased (p<0.005) after infusion of FasL protein-coated TCD-MPB (48±7%, p<0.005) as compared to recipients of naïve TCD-BMC (20±4%) (FIG. 3B).

An additional experiment was performed with lineage-negative MPB (lin⁻MPB) and femoral contents were assayed at 3 days after cell infusion (FIG. 3A). Analysis of femoral bone marrow revealed significant lower fractions (p<0.01) of Jurkat cells (huCD45⁺huCD3⁺) following infusion of lin⁻MPB coated with FasL protein (5.8±1.3%) as compared to naïve lin⁻MPB (12.2±2%) (FIG. 3C). These models document in situ apoptosis of malignant cells due to efficient homing of the therapeutic vehicles to bone marrow, leading to significant reduction in tumor burden.

### Example 4: Human Hematopoietic Progenitors Expressing Ectopic Fas-Ligand Impair Growth of Solid Tumors In Vivo

Direct anti-cancer activity of killer hematopoietic progenitors was further evaluated in solid tumors. Cells were implanted subcutaneously in congenic mice and syngeneic subsets of bone marrow cells were used as vehicles for delivery of the death signals (FIG. 1A). Two prominent examples of prevalent tumors are murine breast (4T1) and colon carcinoma (CT26) implanted in the flank of congenic BALB/c mice (H2K^{d}). Infusion of lineage-negative BMC (lin⁻BMC) expressing FasL protein reduced significantly the growth of breast (FIG. 4A, p<0.05) and colon tumors (FIG. 4B, p<0.005), as compared to infusion of naïve lin⁻BMC. An additional model of murine neuroblastoma (Neuro-2a) was used as an example of a less prevalent tumor for which there is no known effective therapy. Tumors implanted subcutaneously in H2K^{a} mice were inhibited by infusion of T cell-depleted (TCD) BMC overexpressing ectopic FasL protein (FIG. 4C, p<0.01). Notably, all these experiments used syngeneic T cell-depleted and lineage-negative BMC, to emphasize that the hematopoietic progenitors used as carriers of the death signals have significant impact on tumor growth, independent of the activities of immune cells.

We further assessed human colon carcinoma (HT29), one of the prevalent human malignancies, in a surrogate xenogeneic model in immunocompromised NOD.SCID mice using T cell-depleted (TCD) mobilized peripheral blood cells (MPB) as therapeutic vehicles. Grafting of FasL protein-coated human TCD-MPB cells reduced significantly tumor growth rates (FIG. 4D, p<0.05). Notably, human hematopoietic cells do not engraft in immunocompromised NOD.SCID mice without chemical conditioning or radiation, therefore growth inhibition was directly attributed to reduction of malignant cell burden. These data disclose direct activity of therapeutic murine and human hematopoietic progenitors overexpressing FasL protein against solid tumors, without stable engraftment and hematopoietic reconstitution.

### Example 5: Hematopoietic Progenitors Overexpressing Ectopic Fas-Ligand Protein Reduce Metastatic Spread of Melanoma In Vivo

To evaluate the efficacy of killer hematopoietic progenitors in containing metastatic spread, we used a model of murine B16 melanoma implanted in the ear pinna of syngeneic C57Bl/6 mice (H2K^{b}) (FIG. 5A). Surgical excision of the visible tumors usually results in metastatic spread of the malignant cells, which can be identified in the neck lymph nodes of virtually all control mice after surgery. Infusion lineage-negative BMC overexpressing FasL protein at the time of surgery improved survival (10/12) at day 21, as compared to survival of 5/11 controls and 5/10 recipients of naïve lin⁻BMC (FIG. 5B). Furthermore, 4 of the 10 surviving recipients of FasL protein-coated lin⁻BMC showed no evidence of disease at the experimental end point (FIG. 5C). Likewise, treatment with killer cells resulted in reduced incidence of metastases (FIGS. 5D-E): a) 2/6 presented no cervical lymph node metastases as compared to wide spread of melanoma in controls and recipients of naïve lin⁻BMC; b) Reduced incidence of pulmonary metastases in 3/6 mice with lower numbers of 33±7 nodules on lung surface as compared to 4/5 positive controls and recipients of naïve lin⁻BMC with a mean number of 59±13 nodules in each mouse (p<0.005). c) No mice presented liver nodules as compared to 2/5 controls and recipients of naïve cells showing 51±11 mean metastases (p<0.001). Altogether it is demonstrated that infusion of hematopoietic progenitors expressing a death ligand limit significantly the metastatic spread of aggressive tumors, including iatrogenic dissemination at the time of surgery.

### Embodiment 2. Mesenchymal Stromal Cells Overexpressing Death Ligands Induce Apoptosis in Malignant Cells

### Example 6: Mesenchymal Stromal Cells Expressing Ectopic Fas-Ligand Protein Home to and Reduce Growth of Solid Tumors

An additional cell type capable to migrate to and implant in tumors are mesenchymal stromal cells (MSC), which under certain circumstances have significant contribution to vascularization and tumor growth. To demonstrate migration, murine breast carcinoma (4T1) was implanted subcutaneously in immunocompromised NOD.SCID mice and after 3 days were infused with allogeneic MSC derived from the bone marrow of transgenes with constitutive expression of green fluorescent protein (GFP). Migration of MSC to the subcutaneous tumors was detected by in situ microscopy to visualize the fluorescent probe (FIG. 6A), demonstrating MSC affinity to the tumor stroma. To determine the impact of therapy, 4T1 cells were implanted in congenic mice (H2K^{d}) and after 3 days were infused with syngeneic MSC derived from the bone marrow (FIG. 6B). Infusion of naïve syngeneic MSC did not attenuate tumor growth as compared to unmanipulated mice, whereas overexpression of FasL protein decreased significantly the tumor growth rates (FIG. 6C, p<0.05). Reduced tumor burden was associated with better preservation of body weight of mice infused with FasL-coated MSC (FIG. 6D). These data demonstrate that autologous MSC expressing a short-lived protein home to solid tumors and reduce the tumor burden in vivo.

### Example 7: Mesenchymal Stromal Cells Expressing Death Ligands Induce Apoptosis in Human Leukemia Cells In Vivo

Apoptosis-resistant cells, such as mesenchymal stromal cells, may be used to induce apoptosis in leukemic cells found in various compartments. For proof of concept, MSC overexpressing the ectopic FasL protein were directly inoculated into the bone marrow of immunocompromised NOD.SCID mice concomitant with intravenous infusion of relatively large numbers of human Jurkat leukemia/lymphoma B cells (FIG. 7A). Direct intra-bone marrow infusion was used because MSC do not navigate efficiently to the bone marrow following intravenous infusion. The load of Jurkat cells was significantly reduced in mice inoculated with MSC expressing the death ligands (FIG. 7B), demonstrating the efficacy of killer MSC to induce apoptosis and mediate effective elimination of malignant cells in vivo.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for producing modified cells, the method comprises:
(a) providing a sample comprising hematopoietic stem or progenitor cells (HSPCs) or mesenchymal stromal cells (MSCs);
and
(b) adsorbing a death ligand protein to a plasma membrane of said HSPCs or MSCs;
thereby producing therapeutic cells.

2. The method of claim 1, wherein the sample is at least one of:
a. comprising a population of hematopoietic cells and HSPCs are selected from said population;
b. comprising a population of hematopoietic cells and selecting HSPCs comprises selecting a subset of non-immune cells,
c. derived from umbilical cord blood (UCB), bone marrow (BM) or mobilized peripheral blood (MPB);
d. derived from UCB or BM;
e. harvested from mobilized peripheral blood by apheresis;
f. freshly harvested, preserved, or cryopreserved;
g. depleted of immune cells;
h. depleted of T cells;
i. comprising lineage-negative hematopoietic progenitors; and
j. comprising ghosts of HSPCs and MSCs, vesicles from HSPCs and MSCs, or liposomes from HSPCs and MSCs.

3. (canceled)

4. (canceled)

5. (canceled)

6. The method of claim 1, wherein the MSCs are derived from bone marrow, adipose tissue, placenta or umbilical cord.

7. (canceled)

8. (canceled)

9. (canceled)

10. (canceled)

11. (canceled)

12. The method of claim 1, wherein said death ligand protein is at least one of:
a. an exogenous death ligand protein,
b. a member of the tumor necrosis factor (TNF) ligand superfamily;
c. selected from the group consisting of Fas ligand (FasL), TNF-α, and tumor necrosis factor-related apoptosis inducing ligand (TRAIL) and
d. is FasL.

13. (canceled)

14. (canceled)

15. The method of claim 12, wherein said TNF superfamily ligand is FasL.

16. The method of claim 1, wherein said adsorbing comprises at least one of:
a. linking said death ligand protein to said plasma membrane by an exogenous linkage;
b. linking said death ligand protein to said plasma membrane by biotin-streptavidin linkage;
c. providing a fusion protein comprises said death ligand protein and a binding domain that binds a component of said plasma membrane and contacting said fusion protein to said plasma membrane; and
d. providing a death ligand protein comprising a hydrophobic or lipophilic region or moiety and inserting said death ligand protein into said plasma membrane.

17. (canceled)

18. The method of claim 16, comprising non-specifically biotinylating said plasma membrane, providing said death ligand protein coupled to streptavidin and contacting said biotinylated plasma membrane with said provided death ligand protein.

19. The method of claim 16, wherein at least one of:
a. said binding domain binds a protein embedded in said plasma membrane;
b. said binding domain binds a non-proteinaceous component of said plasma membrane;
c. said method comprises providing a fusion protein said death ligand protein and said hydrophobic or lipophilic region or moiety and contacting said provided fusion protein to said plasma membrane;
d. said death ligand protein does not comprise a transmembrane domain; and
e. said HSPCs, MSCs or both do not comprise exogenous DNA or RNA encoding said death ligand protein.

20. (canceled)

21. (canceled)

22. (canceled)

23. (canceled)

24. Modified cells produced by a method of claim 1.

25. A hematopoietic stem or progenitor cell (HSPC) or mesenchymal stromal cell (MSC) having adhered thereto an exogenous death ligand protein.

26. The HSPC or MSC of claim 25, wherein said exogenous death ligand protein is adsorbed to a plasma membrane of said HSPC or MSC.

27. (canceled)

28. (canceled)

29. The HSPC or MSC of claim 25, wherein said HSPC or MSC does not comprise exogenous DNA or RNA encoding said exogenous death ligand.

30. The HSPC or MSC of claim 25, wherein said exogenous death ligand:
a. is not imbedded in a plasma membrane of said HSPC or MSC;
b. is not bound to its native receptor expressed from said HSPC or MSC;
c. is linked by an exogenous linkage to said plasma membrane;
d. is linked by a biotin-streptavidin linkage to said plasma membrane;
e. is part of a fusion protein and said fusion protein comprises a binding domain that binds a component of said plasma membrane; or
f. comprises a hydrophobic or lipophilic region or moiety that is inserted into said plasma membrane.

31. The HSPC or MSC of claim 30, wherein at least one of:
a. said plasma membrane is biotinylated and said exogenous death ligand is coupled to streptavidin;
b. said binding domain binds a protein embedded in said plasma membrane;
c. said binding domain binds a non-proteinaceous component of said plasma membrane; and
d. said exogenous death ligand is part of a fusion protein and said fusion protein comprises said hydrophobic or lipophilic region or moiety.

32. (canceled)

33. (canceled)

34. (canceled)

35. The HSPC or MSC of claim 25, wherein at least one of:
a. said exogenous death ligand protein does not comprise a transmembrane domain,
b. said exogenous death ligand protein is a member of the tumor necrosis factor (TNF) ligand superfamily;
c. said exogenous death ligand is selected from the group consisting of Fas ligand (FasL), TNF-α, and tumor necrosis factor-related apoptosis inducing ligand (TRAIL); and
d. said exogenous death ligand is FasL.

36. (canceled)

37. (canceled)

38. The HSPC or MSC of claim 35, wherein said TNF superfamily ligand is FasL.

39. A composition comprising an HSPC, MSC or both of claim 25.

40. The composition of claim 39, further comprising an acceptable carrier or adjuvant, formulated for systemic administration or intratumoral administration to a subject or wherein said HSPC or MSC is allogeneic, autologous or syngeneic to a subject.

41. (canceled)

42. (canceled)

43. A method of treating cancer in a subject in need thereof, the method comprising: administering to said subject a composition of claim 39, thereby treating a cancer in a subject.

44. The method of claim 43, wherein at least one of:
a. said HSPC, MSC or both are extracted from said subject and said administering comprises returning said HSPCs, MSCs or both to said subject after said exogenous death ligand protein is adhered thereto;
b. said HSPC, MSC or both is irradiated or treated with an agent that arrest differentiation, proliferation or both before said administering;
c. said method further comprises administering at least one other anticancer therapy; and
d. said method further comprises administering at least one of radiotherapy, chemotherapy and immunotherapy.

45. (canceled)

46. (canceled)

47. (canceled)
